SEQUENCE LISTING (1 sequence)

SEQ ID NO: 1 - protein, 299 aa, Artificial Sequence
  Synthetic Construct
FVNQHLCGSHLVEALELVCGERGFHYGGGGGGSGGGGGIVEQCCTSTCSLDQLENYCGGGGGQGGGGQGGGGQGGGGGECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

(19) United States
(12) Patent Application Publication
(10) Pub. No.: US 2024/0082363 A1
(43) Pub. Date: Mar. 14, 2024

(54) METHODS OF TREATING DIABETES

(71) Applicant: Eli Lilly and Company, Indianapolis, IN (US)

(72) Inventors: Molly Corbett CARR, Wayne, PA (US); Emmanuel CHIGUTSA, Zionsville, IN (US); Jenny Y. CHIEN, Indianapolis, IN (US); Parag GARHYAN, Carmel, IN (US); Axel Richard Karl-August HAUPT, Zionsville, IN (US); Cheng Cai TANG, Singapore (SG)

(21) Appl. No.: 18/266,717

(22) PCT Filed: Dec. 14, 2021
(86) PCT No.: PCT/US2021/063235

§ 371 Date: Jun. 12, 2023

Related U.S. Application Data
(60) Provisional application No. 63/125,165, filed on Dec. 14, 2020.

(51) Int. Cl. A61K 38/28; A61K 9/08; A61K 47/02; A61K 47/10; A61K 47/34; A61P 3/10

(52) U.S. Cl. CPC A61K 38/28; A61K 9/08; A61K 47/02; A61K 47/10; A61K 47/34; A61P 3/10

(57) ABSTRACT

Described herein are fixed doses and dosing regimens for long-acting insulin receptor agonists suitable for once-weekly dosing, such as weekly basal insulin-Fc (BIF).

The present invention relates to methods, uses, dosage regimens and products for treating diabetes. More particularly, the present invention relates to methods of treating diabetes with and compositions and products comprising long-acting insulin receptor agonists. The methods described herein include fixed doses and dosing regimens for long-acting insulin receptor agonists suitable for once-weekly dosing, such as weekly basal insulin-Fc (BIF), and product presentations for use in such regimens.

Diabetes is a chronic disorder characterized by hyperglycemia resulting from defects in insulin secretion, insulin action, or both. Type 2 diabetes (T2D) is characterized by elevated blood glucose levels resulting from impaired insulin secretion, insulin resistance, excessive hepatic glucose output, and/or contributions from all of the above. Treatment of patients with T2D typically begins with prescribed weight loss, exercise, and a diabetic diet, but when these measures fail to control elevated blood sugars, then oral medications and incretin-based therapy may be necessary. When these medications are still insufficient, treatment with insulin is considered. T2D patients whose disease has progressed to the point that insulin therapy is required are generally started on a single daily injection of a long-acting, basal insulin.

Basal insulin analogs currently available include insulin glargine, sold under the tradenames LANTUS®, TOUJEO®, BASALGLAR® and SEMGLEE® insulin detemir, sold under the tradename LEVEMIR®, and insulin degludec, sold under the tradename TRESIBA®. These insulins are each indicated for once-daily administration. Many T2D patients are hesitant to initiate and/or comply with insulin therapy, however, due in part to the need for daily injections and dose requirements that require calculation of variable doses on a regular basis. Therefore, even after initiation of insulin therapy, many diabetic patients are unwilling or unable to comply, or are incapable of complying, with the insulin therapy necessary to maintain close control of blood glucose levels.

Research is being conducted to identify insulin products with longer duration of action; thus, requiring fewer injections than currently available insulin products, including as infrequently as once-weekly. For example, WO2014/009316 describes insulin derivatives which are stated to have a long enough time of action that it is sufficient to administer them with a frequency of about once weekly in order for the diabetic patient to get a sufficient basal administration of insulin. A treatment regimen for these derivatives is proposed in WO2016/001185. US2016/0324932 describes fusion proteins having prolonged duration of action at the insulin receptor sufficient for dosing as infrequently as once-weekly, including BIF. A specific dosing regimen is not described.

Despite these disclosures, there remains a need for insulin therapies requiring fewer injections than currently available insulin products and that can be administered with simple and convenient dosing regimens while still providing sufficient glycemic control. There also remains a need for presentations of such therapies that provide for a simpler, more convenient and/or less painful patient experience. There also remains a need for methods of treatment using such insulin therapies either without increasing or with reducing the risk of hypoglycemia compared to currently available insulin products.

### SUMMARY OF THE INVENTION

Accordingly, the present invention provides a method of providing glycemic control in a patient in need thereof with type 2 diabetes (T2D) comprising: administering to said patient once-weekly a fixed dose of BIF selected from the group consisting of 100, 150, 250 and 400 U.

The present invention also provides a method of providing glycemic control in a in a patient with type 2 diabetes (T2D) comprising:
a) administering to said patient an initial dose of 100 U of basal insulin-Fc (BIF) once weekly;
b) increasing the dose to 150 U of BIF once-weekly after at least 4 weeks on the 100 U dose;
c) increasing the dose to 250 U of BIF once-weekly after at least 4 weeks on the 150 U dose; and
d) increasing the dose to 400 U of BIF once-weekly after at least 4 weeks on the 250 U dose.
   In certain embodiments, steps b) through d) are performed when the patient's fasting glucose (FG) is >130 mg/dL.

The present invention also provides an aqueous pharmaceutical composition comprising:
a) a fixed dose of BIF in an amount selected from the group consisting of 100, 150, 250 and 400 U;
b) phosphate in a concentration between about 5 and 10 mM; and
c) glycerol in a concentration between about 15 and 35 mM;
and having a pH between about 5.5 and 7.5.

The present invention also provides a single-use autoinjector for use in improving glycemic control in a patient with T2D comprising a fixed dose of BIF selected from the group consisting of 100, 150, 250 and 400 U.

### DETAILED DESCRIPTION OF THE INVENTION

The present application provides multiple aspects of dosing regimens, uses and methods of treatment for a long-acting insulin receptor agonists suitable for once-weekly dosing, such as those described in U.S. Patent Application Publication No. 2016/0324932, including BIF. In certain aspects, the regimens and methods described herein include administration of a fixed dose of BIF. In other aspects, the regimens and methods described herein include determination of whether the fixed dose of BIF being used to treat a T2D patient should be changed. In other aspects, the regimens and methods described herein include identification of a simple and convenient device for administering a fixed dose of BIF. In other aspects, the regimens and methods herein describe a non-preserved formulation for use in providing a fixed dose of BIF.

BIF, also known as insulin efsitora alfa, comprises a dimer of an insulin receptor agonist fused to a human IgG Fc region, wherein the insulin receptor agonist comprises an insulin B-chain analog fused to an insulin A-chain analog through the use of a first peptide linker and wherein the C-terminal residue of the insulin A-chain analog is directly fused to the N-terminal residue of a second peptide linker, and the C-terminal residue of the second peptide linker is directly fused to the N-terminal residue of the human IgG Fc region. BIF is identified by CAS registry number 2131038-11-2, which provides the following chemical names: (1) Insulin [16-glutamic acid, 25-histidine, 27-glycine, 28-glycine, 29-glycine, 30-glycine] (human B-chain) fusion protein with peptide (synthetic 7-amino acid linker) fusion protein with insulin [47-threonine, 51-aspartic acid, 58-glycine] (human A-chain) fusion protein with peptide (synthetic 20-amino acid linker) fusion protein with immunoglobulin G2 (human Fc fragment), dimer; and (2) Homo sapiens Insulin B-chain [Y16>Y(16), F25>H(25), TPKT27-30>GGGG(27-30)] (1-30) fusion protein with diglycylseryltetraglycyl (31-37) Insulin A-chain [I10>T(47), Y14>D(51), N21>G(58)] (38-58) fusion protein with tris(tetraglycylglutaminyl)pentaglycyl (59-78) Homo sapiens Immunoglobulin heavy constant gamma 2 (del-CH1, hinge-(7-12), CH2, CH3[K¹⁰⁷>del(300)]) (79-299), dimer (80-80′:83-83′)-bisdisulfide, expressed in CHO cells, alfa glycosylated.

Each monomer of BIF has the amino acid sequence set forth in SEQ ID NO:1:

FVNQHLCGSHLVEALELVCGERGFHYGGGGGGSGGGGGIVEQCCTSTCSL
DQLENYCGGGGGQGGGGQGGGGQGGGGGECPPCPAPPVAGPSVFLFPPKP
KDTLMISRTPEVTCVVVDVSHEDPEVQFNWYVDGVEVHNAKTKPREEQFN
STFRVVSVLTVVHODWLNGKEYKCKVSNKGLPAPIEKTISKTKGQPREPQ
VYTLPPSREEMTKNQVSLTCLVKGFYPSDIAVEWESNGQPENNYKTTPPM
LDSDGSFFLYSKLTVDKSRWOOGNVFSCSVMHEALHNHYTOKSLSLSPG

(SEQ ID NO:1). Each monomer includes intrachain disulfide bonds between cysteine residues at positions 7 and 44, 19 and 57, 43 and 48, 114 and 174 and 220 and 278. The two monomers are attached by disulfide bonds between the cysteine residues at positions 80 and 83 to form the dimer. BIF's structure, function and production are described in more detail in U.S. Patent Application Publication No. 2016/0324932.

When used herein, the term “BIF” refers to any insulin receptor agonist comprised of two monomers having the amino acid sequence of SEQ ID NO:1, including any protein that is the subject of a regulatory submission seeking approval of an insulin receptor agonist product that relies in whole or part upon data submitted to a regulatory agency by Eli Lilly and Company relating to BIF, regardless of whether the party seeking approval of said product actually identifies the insulin receptor agonist as BIF or uses some other term.

BIF is a long-acting insulin receptor agonist with a pharmacokinetic and pharmacodynamic profile that is sufficiently prolonged to control blood glucose levels between meals when administered no more frequently than once weekly. Unlike existing insulin therapies, which require individualized and variable dose determinations for a patient's needs at a given point in time, BIF's relatively flat pharmacokinetic profile, with a peak to trough ratio close to 1, allows for its use in simple and convenient dosing regimens comprising administration of a discrete number of fixed doses. Such an approach resembles fixed dose drug therapy in that a limited number of dose strengths are offered and the maximum dose available is capped.

This simplified number of dose offerings is expected to facilitate transition to insulin therapy in patients with T2DM who are naïve to insulin therapy. Such regimens may help alleviate the clinical inertia that limits effective titration of basal insulins in real-world practice. Thus, while the methods, uses and regimens of the present invention may be used to provide glycemic control in a broad range of T2D patients, they are particularly suitable for patients who are not currently being treated with a basal insulin and who are starting on a once-weekly insulin receptor agonist—referred to herein as “insulin-naïve” patients.

In certain embodiments the patient has a glycated hemoglobin Alc (HbA1c) value of 7.0% to 10.0%. In certain embodiments the patient is also being treated with 0 to up to 3 additional antihyperglycemic therapies selected from the group consisting of thiazolidinediones (TZDs), dipeptidyl peptidase IV (DPP4) inhibitors, sodium-glucose co-transporter-2 (SGLT2) inhibitors, biguanides (e.g. metformin), alpha-glucosidase inhibitors, or glucagon-like peptide-1 (GLP-1) receptor agonists. In certain embodiments, the patient has a body mass index (BMI)≤45 kg/m2. In certain embodiments, patients treated with such a fixed dose regimen are insulin naive patients with T2DM with HbA1c between 7.5% and 10.0% inclusive and who are receiving 2 or more oral antihyperglycemic medications with or without GLP-1 RA.

In certain embodiments, patients initiate treatment on an initial dose, and escalate to the next higher dose if needed after treatment with the initial dose for a given period of time. For example, patients may be administered the same fixed dose for multiple weeks, such as 4 weeks, and then have his or her dose escalated to the next fixed dose level if the patient is in need of further glycemic control. In certain embodiments, a patient is considered to be in need of further glycemic control if his or her FG is above a certain level, typically between about 120-140 mg/dL. In certain embodiments, a patient is considered to be in need of further glycemic control if his or her FG is >120 mg/dL. In certain embodiments, a patient is considered to be in need of further glycemic control if his or her FG is >140 mg/dL. In certain preferred embodiments, a patient is considered to be in need of further glycemic control if his or her FG is >130 mg/dL. In certain embodiments, if the patient is in need of further glycemic control after treatment with the highest fixed dose, the patient would transition to treatment with a variable dose regimen outside the scope of the present disclosure.

In certain embodiments, patients may decrease to the previous lower dose if their FG is below a certain level. In certain embodiments, patients may decrease to the previous lower dose if their FG is below a certain level, such as <80 mg/dL. In certain embodiments, patients may discontinue treatment if they experience ≥1 episode of nocturnal hypoglycemia or 2 or more episodes of any hypoglycemia while receiving the lowest dose.

The doses of BIF for use in the methods, uses and regimens of the present invention may be expressed as either insulin units (IU, or U) or mg of BIF. In certain embodiments wherein the dose of BIF is expressed in U, the doses available are between about 50 and about 1050 U. In certain embodiments the doses are between about 100 and 500 U. In certain embodiments, the doses are selected from the group consisting of 100, 150, 200, 250, 300, 350, 400, 450 and 500 U. In certain preferred embodiments the doses available are 100, 150, 250 and 400 U. Because the fixed doses described herein are intended to be provided once-weekly, the units identified for a given dose indicate the total number of units of insulin activity that dose is intended to provide over the course of a week.

A preferred dosing regimen comprised of such doses is set forth in Table 1 below:

| | | |
|---|---|---|
| Initial dose | 100 | U |
| FG Target | 80-130 | mg/dL |

| | |
|---|---|
| Subsequent doses available | 150 U, 250 U, 400 U |
| Dose modification timing | Treat with given dose for at least |
| | 4 weeks before increasing |

### Table 1.

According to the regimen set forth in Table 1, a patient would start on the 100 U initial dose, and then increase to the 150 U dose after treatment for at least 4 weeks if his or her FG is greater than 130 mg/dL. Similarly, the dose for a patient being treated with the 150 U dose would increase to 250 U after treatment for at least 4 weeks on the 150 U dose if his or her FG is greater than 130 mg/dL. Similarly, the dose for a patient being treated with the 250 U dose would increase to 400 U after treatment for at least 4 weeks on the 250 U dose if his or her FG is greater than 130 mg/dL. Finally, patients may decrease to the previous lower dose at any time if their FG is below 80 mg/dL. In addition, patients may discontinue treatment if they experience 21 episode of nocturnal hypoglycemia or 2 or more episodes of any hypoglycemia while receiving the lowest dose.

In certain embodiments wherein the dose of BIF is expressed in mg, the doses available are selected from the group consisting of 1, 1.5, 2, 2.5, 2.85, 3, 3.5, 4, 4.3, 4.5, 5, 5.5, 5.7, 6.5, 7, 7.15, 8, 8.6, 9, 10, 11, 11.45, 12, 12.85, 13, 14, 14.3, 15, 16, 17, 18, 19, 20, 21, 22, 23, 24, 25, 26, 27, 28, 29 and 30 mg. In certain embodiments, the doses available are selected from the group consisting of 1, 1.5, 2, 2.85, 3, 4.3, 4.5, 6, 7.15, 10, 11.45, 12, 12.85 and 14.3 mg. In certain embodiments, the doses available are 2.85, 4.3, 7.15 and 11.45 mg.

When used herein, the term “fixed dose” refers to a specific dose that is available in and administered from a single dosage form. The dosage form for the fixed doses of BIF described herein may be any dosage form available for use in subcutaneous administration of an aqueous solution, such as a vial, cartridge, pen injector or pump, although in preferred embodiments the fixed dose is provided in a single-use autoinjector, such as those described in U.S. Pat. No. 8,734,394.

The term “fixed dose regimen,” when used herein, refers to a treatment regimen that involves a plurality of fixed doses and guidelines for determining which fixed dose should be administered at a given point in time. For example, a fixed dose regimen may comprise a set of multiple fixed doses and guidelines for determining which of those fixed doses should be administered for any given week.

An advantage of the fixed doses and regimens described herein, is that they may be carried out using formulations that do not require the use of preservatives. Whereas existing insulin therapies are typically administered using variable dose regimens with multi-use presentations requiring preserved formulations having sufficient antimicrobial effectiveness to meet regulatory requirements, because the fixed doses described herein are preferably administered from a single-use device, they may be provided in a non-preserved formulation. Use of such a non-preserved formulation may be advantageous to traditional preserved insulin formulations from a stability perspective, as the phenolic preservatives used in existing insulin therapies, such as meta-cresol and phenol, are known to create stability issues with proteins and peptides. Thus, such formulations typically require inclusion of additional excipients to ensure sufficient stability.

The non-preserved formulations for use with the fixed dose regimens of the present invention comprise a fixed dose of BIF, a buffer and a tonicity agent. In certain embodiments, the concentration of BIF is between about 5 and about 25 mg/mL. In certain embodiments, BIF is in a concentration that allows for administration of a fixed dose selected from the group consisting of 100, 150, 250 and 400 insulin units (IU, or U). In certain embodiments, BIF is in an amount selected from the group consisting of 100, 150, 250 and 400 insulin units (IU, or U). In certain embodiments, BIF is in an amount selected from the group consisting of 2.85, 4.3, 7.15 and 11.45 mg. In certain embodiments, the fixed dose of BIF is provided in a 0.5 mL solution having BIF concentration selected from the group consisting of 5.7, 8.6, 14.3 and 22.9 mg/mL.

Examples of buffering agents are phosphates, such as dibasic sodium phosphate, citrate, sodium acetate and tris(hydroxymethyl)aminomethane, or TRIS. In certain embodiments, compositions of the present invention include a citrate buffer in a concentration ranging from about 5 to about 10 mM. In certain embodiments, compositions of the present invention include phosphate in a concentration ranging from about 5 to about 10 mM. In certain preferred embodiments, compositions of the present invention include phosphate in a concentration of about 5, 6, 7, 8, 9 or 10 mM. In certain embodiments the buffer is about 10 mM.

Typical tonicity agents include glycerol (glycerin), mannitol and sodium chloride. If the addition of a tonicity agent is required, glycerin is preferred. In certain embodiments the concentration of glycerol is from about 10 to about 50 mg/mL. In certain embodiments the concentration of glycerol is from about 15 to about 35 mg/mL. In certain embodiments the concentration of glycerol is selected from the group consisting of about 15, 17, 20, 21 and 35 mg/mL. In certain preferred embodiments, the concentration of glycerin is about 25 mg/mL.

In certain embodiments, the composition has a pH from about 5.5 to about 7.5, preferably at least about 6.1. In certain embodiments, the pH ranges from about 6.2 to about 7.4. In certain preferred embodiments, the pH ranges from about 6.3 to about 6.9. In a particularly preferred embodiment, the pH is about 6.5.

The compositions may also include other excipients, including stabilizing agents such as surfactants. Examples of surfactants disclosed for use in parenteral pharmaceutical compositions include polysorbates, such as polysorbate 20 (TWEEN®20) and polysorbate 80 (TWEEN 80), polyethylene glycols such as PEG 400, PEG 3000, TRITON™ X-100, polyethylene glycols such as polyoxyethylene (23) lauryl ether (CAS Number: 9002-92-0, sold under trade name BRIJ®), alkoxylated fatty acids, such as MYRJ™, polypropylene glycols, block copolymers such as poloxamer 188 (CAS Number 9003-11-6, sold under trade name PLURONIC® F-68) and poloxamer 407 (PLURONIC® F127), sorbitan alkyl esters (e.g., SPAN®), polyethoxylated castor oil (e.g., KOLLIPHOR®, CREMOPHOR®) and trehalose and derivatives thereof, such as trehalose laurate ester.

In certain embodiments, the composition comprises a surfactant selected from the group consisting of polysorbate 20, polysorbate 80 and poloxamer 188. Most preferred is poloxamer 188. In certain embodiments, the concentration of surfactant ranges from about 0.01 to about 10 mg/mL or about 0.1 to about 0.5 mg/mL. In preferred embodiments wherein the surfactant is poloxamer 188, the concentration of poloxamer 188 is about 0.4 mg/mL.

In certain embodiments, the composition comprises BIF in a concentration between 2.5 and 25 mg/mL, a buffer, and a tonicity agent, and has a pH from 5.5 to 7.5. In certain embodiments, the composition comprises BIF in an amount between 100-400 U. In certain embodiments, the composition comprises BIF in an amount selected from the group consisting of about 100, 150, 250 or 400 U. In certain embodiments, the buffer is a phosphate buffer in a concentration between 5 and 10 mM, the tonicity agent is glycerol in a concentration from 15-35 mg/mL and the pH is between 6.3-6.9. In certain embodiments, the composition further comprises a surfactant. In certain embodiments the surfactant is poloxamer 188 in a concentration between 0.01 to 10 mg/mL. In certain embodiments, the composition comprises BIF in an amount selected from the group consisting of about 100, 150, 250 or 400 U, phosphate in a concentration of about 10 mM, glycerol in a concentration of about 25 mg/mL, poloxamer 188 in a concentration of about 0.4 mg/mL and has a pH of about 6.5. In certain embodiments, the composition contains no preservative(s). In certain embodiments, the composition does not contain zinc as a stabilizing agent.

Preferred compositions have chemical and physical stability is sufficient to allow for storage of at least 24 months at 5° C. and at least 2 weeks of storage at temperatures up to 30° C. without loss of stability. In certain embodiments, the compositions are sufficiently stable to allow for storage for 8 weeks at 25° C. In certain embodiments, the compositions are sufficiently stable to allow for storage for 12 weeks at 25° C. In certain embodiments, the compositions are sufficiently stable to allow for storage for 8 weeks at 30° C. In certain embodiments, the compositions are sufficiently stable to allow for storage for 12 weeks at 30° C.

When used herein, the terms “approximately” and “about” are intended to refer to an acceptable degree of error for the amount or quantity indicated given the nature or precision of the measurements. For example, the degree of error can be indicated by the number of significant figures provided for the measurement, as is understood in the art, and includes but is not limited to a variation of +/−1 in the most precise significant figure reported for the amount or quantity. Typical exemplary degrees of error are within 20 percent (%), preferably within 10%, and more preferably within 5% of a given value or range of values. Numerical quantities given herein are approximate unless stated otherwise, meaning that the term “about” can be inferred when not expressly stated.

When used herein, the term “dose” or “doses” refers to the quantity of insulin receptor agonist suitable for once weekly dosing that is administered to an individual in discrete amount at a particular point in time.

When used herein, the terms “fasting glucose,” “FG,” “fasting blood glucose,” “FBG,” “fasting plasma glucose” or “FPG” refer to plasma glucose level from a sample of blood taken or obtained via continuous glucose monitoring (CGM) after a patient fasts overnight. When used in the context of determining the dose of insulin receptor agonist suitable for once weekly dosing to be administered to a patient, unless otherwise specified herein, the patient's FG is determined as the median FG from multiple days, typically at least 3 days and no more than 7 days.

When used herein, the terms “treatment,” “treat,” “treating,” and the like, are meant to include slowing or attenuating the progression of a disease or disorder. These terms also include alleviating, ameliorating, attenuating, eliminating, or reducing one or more symptoms of a disorder or condition, even if the disorder or condition is not actually eliminated and even if progression of the disorder or condition is not itself slowed or reversed.

“Glycemic control” refers to a subject's blood sugar levels, as measured for example by blood glucose and/or HbA1c levels; “providing” glycemic control refers to maintaining or improving glycemic control; “maintaining” glycemic control refers to maintaining the time with blood glucose levels within a target range and/or maintaining or reducing HbA1c; “improving” glycemic control refers to increasing the time with blood glucose levels within a target range and/or reductions in HbA1c; and “in need of further” glycemic control refers to a need for an increased time with blood glucose levels in a target range and/or reductions in HbA1c.

“HbA1c” refers to glycated hemoglobin levels, which develop when hemoglobin joins with glucose in the blood. HbA1c levels are a commonly used measure of glycemic control in patients with diabetes.

“Hypoglycemia” refers to low blood sugar, and an “episode” of hypoglycemia refers to an instance of low blood sugar, as observed for example in a plasma glucose test or value from a personal blood glucose meter (BGM) or CGM device, in many cases less than about 70 mg/dL.

An episode of “severe” hypoglycemia is a severe event characterized by altered mental and/or physical status requiring assistance for treatment of hypoglycemia. For example, a subject with altered mental status, and could not assist in their own care, or was semiconscious or unconscious, or experienced coma with or without seizures, and the assistance of another person was needed to actively administer carbohydrate, glucagon, or other resuscitative actions. Glucose measurements may not be available during such an event, but neurological recovery attributable to the restoration of glucose concentration to normal is considered sufficient evidence that the event was induced by a low glucose concentration.

The methods of treatment, regimens and uses described herein may be provided in simultaneous or sequential combination with other T2D treatments, including oral T2D medications such as metformin, incretins and/or other injectable medications. Examples of incretins include GLP-1 receptor agonists, such as dulaglutide or semaglutide, GIP/GLP-1 co-agonists such as tirzepatide, and GIP/GLP-1/glucagon triple agonists. In certain embodiments, the methods of treatment, regimens and uses described herein may be provided in simultaneous or sequential combination with other basal insulins and/or rapid-acting insulins.

Certain embodiments of the methods, uses and treatments described herein are as follows:

Embodiment 1. A method of improving glycemic control in a subject having diabetes comprising:
a) identifying a subject having diabetes;
b) administering to said subject a first dose of an insulin receptor agonist suitable for one weekly dosing for one or more weeks;
C) measuring the subject's FG multiple times during the week following the most recent administration of the first dose;
d) determining whether the subject's median FG from the measurements described in step c) was >130 mg/dL;
e) selecting the next dose of insulin receptor agonist to be administered according to the following criteria: (i) if the subject's FG was >130 mg/dL, switching the subject to a second dose of insulin receptor agonist suitable for once weekly dosing; or (ii) if the subject's FG was <130 mg/dL, maintaining the subject on the first dose of insulin receptor agonist suitable for once weekly dosing; and
f) administering to the subject the dose selected in step e).

Embodiment 2. The method of embodiment 1, wherein the dose of insulin receptor agonist administered in step f) is the second dose of insulin receptor agonist suitable for once weekly dosing, and wherein the second dose of insulin receptor agonist suitable for once weekly dosing has been administered for one or more weeks; and further comprising:
g) measuring the subject's FG multiple times during the week following the most recent administration of the second dose;
h) determining whether the subject's median FG from the measurements described in step g) was >130 mg/dL; and
i) selecting the next dose of insulin receptor agonist to be administered according to the following criteria: (i) if the subject's FG was >130 mg/dL, switching the subject to a third dose of insulin receptor agonist suitable for once weekly dosing; (ii) if the subject's FG was 81-130 mg/dL, maintaining the subject on the second dose of insulin receptor agonist suitable for once weekly dosing; or (iii) if the subject's FG was <81, switching the subject to the first dose of insulin receptor agonist suitable for once weekly dosing; and
j) administering to the subject the dose selected in step i).

Embodiment 3. The method of embodiment 2, wherein the dose of insulin receptor agonist administered in step j) is the third dose of insulin receptor agonist suitable for once weekly dosing, wherein the third dose of insulin receptor agonist suitable for once weekly dosing has been administered for one or more weeks; and further comprising:
k) measuring the subject's FG multiple times during the week following the most recent administration of the third dose;
l) determining whether the subject's median FG from the measurements described in step k) was >130 mg/dL;
m) selecting the next dose of insulin receptor agonist to be administered according to the following criteria: (i) if the subject's FG was >130 mg/dL, switching the subject to a fourth dose of insulin receptor agonist suitable for once weekly dosing; (ii) if the subject's FG was 81-130 mg/dL, maintaining the subject on the third dose of insulin receptor agonist suitable for once weekly dosing; or (iii) if the subject's FG was <81, switching the subject to the second dose of insulin receptor agonist suitable for once weekly dosing; and
n) administering to the subject the dose selected in step m).

Embodiment 4. The method of embodiment 3, wherein the dose of insulin receptor agonist administered in step n) is the fourth dose of insulin receptor agonist suitable for once weekly dosing, wherein the fourth dose has been administered for one or more weeks; and further comprising:
o) measuring the subject's FG multiple times following the most recent administration of the fourth dose;
p) determining whether the subject's median FG from the measurements described in step o) was >140 mg/dL for at least two consecutive weeks;
q) selecting the next dose of insulin receptor agonist to be administered according to the following criteria: (i) if the subject's FG was >140 mg/dL for two consecutive weeks, determining the next dose to be administered according to the criteria set forth in any of claims 11-17, 23-24, 30, 32 or 36, (ii) if the subject's FG was <81 following administration of the most recent dose, switching the subject to the third dose of insulin receptor agonist suitable for once weekly dosing; or (iii) if the subject's FG was >81 following administration of the most recent dose and was not >140 mg/dL for two consecutive weeks, maintaining the subject on the fourth dose of insulin receptor agonist suitable for once weekly dosing; and
r) administering to the subject the dose selected in step q).

Embodiment 5. The method of any of embodiments 1-4, wherein the patient discontinues treatment if the patient experiences one or more episodes of nocturnal hypoglycemia or 2 or more episodes of hypoglycemia while receiving the first dose.

Embodiment 6. The method of any of embodiments 1-5 wherein the patient has T2DM.

Embodiment 7. The method of any of embodiments 1-6 wherein the patient is insulin-naïve.

Embodiment 8. The method of any of embodiments 1-7 wherein patient has uncontrolled hyperglycemia.

Embodiment 9. The method of any of embodiments 1-8, wherein the patient has HbA1c between 7.5 and 10.0/o.

Embodiment 10. The method of any of embodiments 1-9, wherein the patient is receiving 2 or more oral antihyperglycemic medications.

Embodiment 11. The method of any of embodiments 1-10 wherein the patient is receiving a GLP-1 receptor agonist.

Embodiment 12. The method of any of embodiments 1-11 wherein the insulin receptor agonist suitable for once weekly dosing is BIF.

Embodiment 13. The method of embodiment 12 wherein the first dose of BIF is 1.5 mg.

Embodiment 14. The method of any of embodiments 1-13 wherein the second dose of BIF is 3.0 mg.

Embodiment 15. The method of any of embodiments 1-14 wherein the third dose of BIF is 4.5 mg.

Embodiment 16. The method of any of embodiments 1-15 wherein the fourth dose of BIF is 6.0 mg.

Embodiment 17. A method of improving glycemic control in a patient having diabetes comprising administering a fixed dose of BIF selected from the group consisting of 1.5, 3.0, 4.5 and 6.0 mg.

Embodiment 18. The method of any of embodiments 1-17 wherein the dose of insulin receptor agonist has been administered for at least 4 weeks before selecting the next dose of insulin receptor agonist to be administered.

Embodiment 19. A method of providing glycemic control in a subject having diabetes and in need of further glycemic control, comprising:
a) identifying a subject in need of further glycemic control;
b) administering to said subject a first dose of an insulin receptor agonist suitable for once weekly dosing for a minimum of four weeks;
c) determining whether the subject is in need of further glycemic control; and if the subject is in need of further glycemic control as determined in step c), administering to said subject a second dose of the insulin receptor agonist suitable for once weekly dosing for a minimum of four weeks.

Embodiment 20. The method of embodiment 19, wherein the subject has been administered the second dose of insulin receptor agonist suitable for once weekly administration for a minimum of four weeks, further comprising:
d) determining whether the subject is in need of further glycemic control; and
e) if the subject is in need of further glycemic control, administering to said subject a third dose of insulin receptor agonist suitable for once weekly dosing for a minimum of four weeks.

Embodiment 21. The method of embodiment 20, wherein the subject has been administered the third dose of insulin receptor agonist suitable for once weekly for a minimum of four weeks, further comprising:
f) determining whether the subject is in need of further glycemic control; and
g) if the subject is in need of further glycemic control, administering to said subject a fourth dose of once weekly for a minimum of two weeks.

Embodiment 22. The method of any of embodiments 19-21 wherein the insulin receptor agonist is BIF.

Embodiment 23. The method of embodiment 22 wherein the first dose is 1.5 mg.

Embodiment 24. The method of embodiment 23 wherein the second dose is 3.0 mg.

Embodiment 25. The method of embodiment 24 wherein the third dose is 4.5 mg.

Embodiment 26. The method of embodiment 25 wherein the fourth dose is 6.0 mg.

Embodiment 27. The method of embodiment 26, wherein the subject has been administered 6.0 mg of BIF once weekly for a minimum of two weeks, further comprising:
h) determining whether the subject is in need of further glycemic control; and
i) if the subject is in need of further glycemic control, administering to said subject a dose of BIF determined according to the criteria set forth in any of claims 11-17, 23-24, 30, 32 or 36.

Embodiment 28. A method of providing glycemic control in a patient in need thereof with type 2 diabetes (T2D) comprising: administering to said patient once-weekly a fixed dose of basal insulin-Fc (BIF) selected from the group consisting of 100, 150, 250 and 400 U.

Embodiment 29. The method of embodiment 28, wherein the first dose of BIF administered to the patient is 100 U.

Embodiment 30. The method of either of embodiments 28 or 29 wherein the patient is administered the same dose for at least 4 weeks, and wherein the dose is increased when the patient needs additional glycemic control.

Embodiment 31. The method of any of embodiments 28-30 wherein the patient's dose is increased if the patient's FG is >130 mg/dL after treatment with a first fixed dose for at least 4 weeks.

Embodiment 32. The method of either of embodiments 30 or 31, wherein the patient's dose is only increased if the patient had 0 episodes of blood glucose <70 mg/dL.

Embodiment 33. The method of any of embodiments 28-32 wherein the patient's dose is decreased if the patient's FG is <80 mg/dL.

Embodiment 34. A method of improving glycemic control in a patient in need thereof with type 2 diabetes (T2D) comprising:
a) administering to said patient an initial dose of 100 U of basal insulin-Fc (BIF) once weekly;
b) increasing the dose to 150 U of BIF once-weekly after at least 4 weeks on the 100 U dose;
c) increasing the dose to 250 U of BIF once-weekly after at least 4 weeks on the 150 U dose; and
d) increasing the dose to 400 U of BIF once-weekly after at least 4 weeks on the 250 U dose.

Embodiment 35. The method of embodiment 34 wherein steps b) through d) are performed to reduce the patient's fasting glucose (FG).

Embodiment 36. The method of embodiment 34 wherein steps b) through d) are performed when the patient's FG is >130 mg/dL.

Embodiment 37. The method of any of embodiments 34-36 wherein steps b) through d) are only performed when the patient had 0 episodes of FG <70 mg/dL.

Embodiment 38. The method of any of embodiments 34-37 wherein the patient's dose is decreased to the previous dose if the patient's FG is <80 mg/dL.

Embodiment 39. The method of any of embodiments 28-38, wherein the patient discontinues treatment if the patient experiences one or more episodes of nocturnal hypoglycemia or 2 or more episodes of hypoglycemia.

Embodiment 40. The method of any of embodiments 28-39 wherein the patient is insulin-naïve.

Embodiment 41. The method of any of embodiments 28-40 wherein patient has uncontrolled hyperglycemia.

Embodiment 42. The method of any of embodiments 28-41, wherein the patient has HbA1c between 7 and 10%.

Embodiment 43. The method of any of embodiments 28-42, wherein the patient is receiving 2 or more oral antihyperglycemic medications.

Embodiment 44. The method of any of embodiments 28-43 wherein the patient is receiving a GLP-1 receptor agonist.

Embodiment 45. The method of any of embodiments 28-44 wherein BIF is in administered in an aqueous composition comprising: phosphate in a concentration between about 5 and 10 mM; and glycerol in a concentration between about 15 and 35 mM; and having a pH between about 5.5 and 7.5.

Embodiment 46. The method of embodiment 45, wherein the composition further comprises poloxamer 188 in a concentration between about 0.1 to about 0.5 mg/mL.

Embodiment 47. The method of embodiment 46 wherein the phosphate is in a concentration of about 10 mM; glycerol is in a concentration of about 25 mM; and poloxamer 188 is in a concentration of about 0.4 mg/mL; and wherein the pH of the composition is about 6.5.

Embodiment 48. The method of any of embodiments 1-47 wherein the method comprises improving glycemic control in the patient.

Embodiment 49. An aqueous pharmaceutical composition comprising:
a) a fixed dose of BIF in an amount selected from the group consisting of 100, 150, 250 and 400 U;
b) phosphate in a concentration between about 5 and 10 mM; and
c) glycerol in a concentration between about 15 and 35 mM;
and having a pH between about 5.5 and 7.5.

Embodiment 50. The composition of embodiment 49, further comprising poloxamer 188 in a concentration between about 0.1 to about 0.5 mg/mL.

Embodiment 51. The composition of embodiment 50 wherein the phosphate is in a concentration of about 10 mM; glycerol is in a concentration of about 25 mM; and poloxamer 188 is in a concentration of about 0.4 mg/mL; and wherein the pH of the composition is about 6.5.

Embodiment 52. A method of improving glycemic control in a patient in need thereof with type 2 T2D comprising administering to said subject the composition of any of embodiments 49-51.

Embodiment 53. The method of any of embodiments 28-48 wherein the fixed dose of BIF is provided in a single-use autoinjector.

Embodiment 54. A single-use autoinjector for use in the method of any of embodiments 28-48.

Embodiment 55. A single-use autoinjector comprising the composition of any of embodiments 49-51.

Embodiment 56. BIF for use in treating diabetes according to the method of any of the above embodiments.

Embodiment 57. Use of BIF in the manufacture of a medicament for use in the treatment of diabetes according to any of the above embodiments.

Embodiment 58. The composition of any of embodiments 49-51, wherein the composition does not comprise a preservative.

Embodiment 59. The composition of any of embodiments 49-51 or 58, wherein the composition does not comprise zinc.

Embodiment 60. The composition of any of embodiments 49-51 or 58-59, wherein the composition does not comprise any additional stabilizing agents.

Embodiment 61. The composition of any of embodiments 49-51 or 58-60, wherein the composition has chemical and physical stability is sufficient to allow for storage of at least 24 months at 5° C. and at least 2 weeks of storage at temperatures up to 30° C. without loss of stability.

Embodiment 62. The composition of any of embodiments 49-51 or 58-61, wherein the composition is sufficiently stable to allow for storage for 8 weeks at 25° C.

Embodiment 63. The composition of any of embodiments 49-51 or 58-62, wherein composition is sufficiently stable to allow for storage for 12 weeks at 25° C.

Embodiment 64. The composition of any of embodiments 49-51 or 58-63, wherein the composition is sufficiently stable to allow for storage for 8 weeks at 30° C.

Embodiment 65. The composition of any of embodiments 49-51 or 58-64, wherein the composition is sufficiently stable to allow for storage for 12 weeks at 30° C.

The invention is further illustrated by the following examples, which are not to be construed as limiting.

### EXAMPLES

### Clinical Studies

Modeling and simulation approaches utilizing Phase 1 and 2 clinical data are used to develop fixed dosing regimens in the form of fixed doses in autoinjectors for Phase 3 clinical evaluations.

A Phase 3, parallel-design, open-label, randomized control trial is designed to evaluate the efficacy and safety of BIF using a fixed dose regimen compared to glargine in patients with T2D who are on background oral anti-hyperglycemic medications, with or without GLP-1 RA, prior to entering the study. Participants will continue prior stable therapy with up to 3 allowed noninsulin diabetes medications during the study.

Participants will be randomly assigned treatment on a 1:1 ratio to receive either BIF once weekly via subcutaneous administration using prefilled auto injector insulin pens, or once daily glargine administered using KwikPen devices. Autoinjectors will be available as 100 U, 150 U, 250 U and 400 U single dose devices. In both the treatment arms, participants will be provided with glucometers for self-monitoring of blood glucoses, instructed about hypoglycemia recognition and treatment, and trained on protocol-related tasks. Participants randomized to BIF will start with an initial dose of 100 units/week for 4 weeks and will sequentially be moved up to the next dose every 4 weeks if target fasting glucose of 80-130 mg/dL is not attained with each dose. If the target glucose is not attained after 4 weeks using the final autoinjector dosing pen (400 units/week), patients will be transitioned to KwikPen containing BIF (which can be used to administer higher and flexible doses).

Investigators will determine participant's daily glargine insulin dose according to protocol and oversee the dose adjustments to achieve similar blood glucose target (80-130 mg/dL) while avoiding hypoglycemia. Study participants will continue the protocol-assigned treatment for a total of 52 weeks and the primary endpoint analysis is expected at 52 weeks.

Key design features are set forth below in Table 2.

TABLE 2
Key design features.

| | Design and feature | Rationale |
|---|---|---|
| Study | 52 weeks | This duration provides |
| duration | | sufficient time for glucose |
| | | levels to stabilize on the |
| | | study insulin and would |
| | | also allow evaluation of |
| | | efficacy and safety with |
| | | long-term use. |
| Comparator | Glargine | It is a well-characterized |
| | | basal insulin administered |
| | | as a once-daily injection. |
| Patient | Patients with T2D ≥18 years of age at | Criteria will allow |
| population | screening | assessment of BIF |
| | Patients on a maximum of 3 anti- | (compared with glargine) |
| | hyperglycemic medications (OAMs +/− | in adult PwT2D that are |
| | GLP-1RA (patients need to be on stable | insulin naïve with |
| | doses at least 3 months prior to screening | glycemic control that |
| | and be willing to continue stable dosing | ranges from fairly good to |
| | throughout the study.) | poor at baseline. |
| | Body mass index (BMI) ≤45 kg/m2, with | |
| | no significant weight gain or loss in the | |
| | past 3 months (>5%). | |
| | HbA1c 7.0%-10% inclusive | |
| Exposure | N = 670 | Approximately 670 |
| | BIF = 335, glargine = 335 | participants will be |
| | | randomized to BIF and |
| | | glargine in 1:1 ratio. With |
| | | the assumption of 15% |
| | | drop out at Week 52, |
| | | approximately 285 |
| | | participants will complete |
| | | for both BIF and glargine |
| | | treatments, at 52 weeks. |
| | | The primary objective is to |
| | | show a NI based on a 0.4% |
| | | margin in HbA1c. |
| | | However, the study is |
| | | powered for a 0.3% NI |
| | | margin to meet global |
| | | regulatory requirements. |
| | | Using a 2-sided 0.05 level |
| | | test and assuming 1.1% |
| | | SD, 0.3% NI margin, and |
| | | no treatment difference for |
| | | HbA1c change from |
| | | baseline, this sample size |
| | | will provide at least 90% |
| | | statistical power to show |
| | | noninferiority of BIF to |
| | | glargine. to glargine. |
| Method of | For BIF, a weekly fixed dose will be | The BIF autoinjectors will |
| delivery | delivered by an autoinjector delivery | be similar to the |
| | device. There are four fixed doses | autoinjectors used for |
| | delivered by AI. If the final dose of BIF | TRULICITY. |
| | does not get the participant into the | KwikPen(s) will be similar |
| | fasting glucose target range of 80-130 | to the BASAGLAR |
| | mg/dL, the participant will be moved to a | KwikPen that has been |
| | KwikPen for BIF dosing. | adapted to deliver a weekly |
| | KwikPen Insulin Delivery device for | dose of BIF in 5-unit |
| | Glargine | increments. |
| | | Glargine will be |
| | | administered using the |
| | | marketed BASAGLAR |
| | | KwikPen. |
| Special | SMBG 6-point profiles will be collected at | 6-Point profiles will |
| procedures | specific timepoints throughout the study | provide an insight into |
| or | | daily glycemic control at |
| diagnostics | | suitable study timepoints. |
| needed | | |
| Dosing | Participants in BIF treatment arm will start | Emerging data with BIF |
| regimen | with an initial dose of 100 units/week for 4 | and PK/PD modeling have |
| | weeks and will sequentially be moved up to | guided selection of |
| | the next dose every 4 weeks if target | autoinjector BIF strengths |
| | fasting glucose of 80-130 mg/dL is not | and will guide dose |
| | attained with each dose. If the target | titrations beyond 400 |
| | glucose is not attained with the final | units/week (if necessary). |
| | autoinjector dosing pen (400 units/week), | |
| | patients will be transitioned to KwikPen | |
| | (which can be used to administer higher | |
| | and flexible BIF doses). | |
| | Instructions for initiating and titrating | |
| | glargine will follow the prescribing | |
| | information and standards of care for once- | |
| | daily insulin glargine. | |
| | Participants may decrease to the previous | |
| | lower dose if FG is below 80 mg/dL and | |
| | may discontinue treatment if they | |
| | experience ≥1 episode of nocturnal | |
| | hypoglycemia or 2 or more episodes of any | |
| | hypoglycemia while receiving the lowest | |
| | dose | |

Abbreviations: BG = blood glucose; FBG = fasting blood glucose; FDA = Food and Drug administration; GLP-1 RA = glucagon-like peptide-1 receptor agonist; HbA1c = glycated hemoglobin A1c; NI = noninferiority; OAM = oral antihyperglycemic medication; PD = pharmacodynamics; PK = pharmacokinetics; PwT2D = people with type 2 diabetes mellitus; SD = standard deviation; SMBG = self-monitored blood glucose.

Inclusion criteria include the following: 1. Are at least 18 years of age at screening (or older per local regulations); 2. Have a diagnosis of type 2 diabetes mellitus (T2D) according to the WHO criteria and not treated with insulin; 3. Have a baseline glycated hemoglobin Alc (HbA1c) value of 7.0% to 10.0%, inclusive, at screening; 4. Acceptable noninsulin diabetes therapies may include 0 to up to 3 of the following: thiazolidinediones (TZDs); dipeptidyl peptidase IV inhibitors; sodium-glucose co-transporter-2 inhibitors; biguanides (e.g. metformin); alpha-glucosidase inhibitors, or glucagon-like peptide-1 receptor agonists (NOTE: All noninsulin diabetes therapies must be used in accordance with the corresponding local product label at the time of screening, and participants should be willing to continue stable dosing throughout the study according to the protocol. Patients need to be on stable doses at least 3 months prior to screening and be willing to continue stable dosing throughout the study); 5. Is insulin naïve, or has been treated with short term insulin treatment for a maximum of 14 days prior to the day of screening and/or prior insulin treatment for gestational diabetes; and 6. Have a BMI ≥45 kg/m2 at screening with no significant weight gain or loss in the past 3 months (≥5%).

Exclusion criteria include the following: 1. Have a diagnosis of type 1 diabetes mellitus or latent autoimmune diabetes, or specific type of diabetes other than T2D (e.g., monogenic diabetes, diseases of the exocrine pancreas, drug-induced or chemical-induced diabetes); 2. Have received any of the following nonallowed diabetes medication within 30 days prior to screening including glinides, pramlintide, sulfonylureas, insulin; 3. Have a diagnosis of type 1 diabetes mellitus or latent autoimmune diabetes, or specific type of diabetes other than T2D (e.g., monogenic diabetes, diseases of the exocrine pancreas, drug-induced or chemical-induced diabetes); 4. Have received any of the following nonallowed diabetes medication within 30 days prior to screening including glinides, pramlintide, sulfonylureas, insulin; 5. Have any other serious disease or condition (for example, known drug or alcohol abuse or psychiatric disorder) that, in the opinion of the investigator, would pose a significant risk to the study participant, preclude the study participant from following and completing the protocol; 6. Hematologic: have had a blood transfusion or severe blood loss within 3 months prior to Visit 1 or have known hemoglobinopathy, hemolytic anemia or sickle cell anemia, or any other traits of hemoglobin abnormalities known to interfere with the measurement of HbA1c in the opinion of the investigator; 7. Are receiving chronic (>14 days) systemic glucocorticoid therapy (excluding topical, intraocular, intranasal, or inhaled preparations) or have received such therapy for >14 days within the month preceding screening.

Efficacy and safety objectives, assessments and endpoints are set forth in Table 3 below.

TABLE 3

| | |
|---|---|
| Objectives | Primary: |
| | To demonstrate noninferiority to glargine for the change in |
| | HbA1c from baseline to 52 weeks, with a noninferiority |
| | margin of 0.4%. |
| | Key Secondary: |
| | Change in HbA1c from baseline at week 52 |
| | Event rate of participant-reported clinically significant |
| | nocturnal hypoglycemia (<54 mg/dL or severe) from |
| | baseline to week 52 |
| Key safety | Adverse events |
| assessments | Hypoglycemia |
| | Allergic/hypersensitivity reactions |
| | Immunogenicity |

As seen in Table 3, the primary efficacy measurement is HbA1c, a widely used measure of glycemic control that reflects a cumulative history of glucose levels in the preceding 2 to 3 months. It has been found to correlate well with the risk of long-term diabetes complications. It is a well-accepted measure in assessing a drug's glucose-lowering efficacy. The other secondary objectives provide complementary information about glycemic control. Hypoglycemia, adverse events, and immunogenicity will be assessed to characterize safety.

A similar study to that described above is designed to study another exemplary regimen including 4 fixed doses in mg (e.g., 1.5, 3.0, 4.5, 6.0 mg). Such a presentation is designed for insulin-naïve patients with T2DM being treated with oral or injectable antidiabetic medications. A Phase 3 study is designed to evaluate these fixed doses. The study population includes insulin naive patients with T2DM with uncontrolled hyperglycemia (e.g. HbA1c between 7.5% and 10.0% inclusive) who are receiving 2 or more oral antihyperglycemic medication with or without injectable GLP-1 RA.

The primary objective is to demonstrate noninferiority of fixed dose BIF on glycemic control compared with insulin glargine in insulin naive patients with T2DM who are receiving oral antihyperglycemic medications with or without injectable GLP-1 receptor agonists.

Patients are randomized to receive fixed dose BIF or individualized doses of insulin glargine. Patients who are randomized to fixed dose BIF will start treatment with the lowest dose (for example, 1.5 mg/week) and will escalate to higher doses every 4 weeks if needed. The current modeling results suggest that doses can be escalated when the median FG is >130 mg/dL. Patients may decrease to the previous lower dose if median FPG is <80 mg/dL. Patients will discontinue treatment if they experience ≥1 episode of nocturnal hypoglycemia or 2 or more episodes of any hypoglycemia while receiving the lowest dose.

Patients who still need additional glycemic control while receiving the highest fixed dose (for example 6 mg/week) may transition to a variable dose paper algorithm. For example, participants having FG >140 mg/dL for 2 consecutive weeks while receiving the highest fixed dose may be transitioned to a variable dose algorithm as described above. Safety measures will be similar to those described in the variable dose algorithm studies described above.

The results for this treatment regimen will be compared to insulin glargine treatment based on a standard of care, treat-to-target algorithm used with that product. This design will enable the evaluation of a simplified weekly fixed dose option for patients with T2DM who need to initiate basal insulin while limiting the complexity related to daily titration of insulin doses. This design will also enable evaluation of the transition between the highest fixed dose to the paper algorithm for those who need additional glycemic control.

The results of simulations support that in insulin naïve patients with T2DM, BIF can achieve a comparable benefit-risk profile with 3 to 4 dose strengths compared to a once-daily basal insulin adjusted according to standard, unrestricted titration regimens. As an easy starter weekly basal insulin, BIF is expected to provide noninferior efficacy and hypoglycemia rates compared to insulin glargine. The four selected dose levels for the fixed dose approach reasonably approximate the corresponding glargine doses used by the majority of patients in this population.

### Formulation Study

A study is designed to test the stability of non-preserved BIF drug product at a range of doses across those described herein. Compositions are prepared containing BIF in concentrations of 2.5 mg/mL and 25 mg/mL, phosphate buffer at a concentration of 10 mM, glycerin at a concentration of 25 mg/mL and poloxamer 188 at a concentration of 0.4 mg/mL and having a pH of 6.5+/−0.2. Samples are prepared by filling 0.5 mL of the compositions into semi-finished syringes and stored at one of four storage conditions for up to 24 months: 5° C.; 25° C./60% relative humidity (RH); 0.5° C.; 30° C./65% RH.

Samples are withdrawn at timepoints of 0, 1, 3, 6, 9, 12, 18 and 24 months and analyzed by various stability indicating assays, including: in vitro potency, size exclusion chromatography (SEC) (purity, aggregates, fragments), RP-HPLC (main peak purity, related substances), anion exchange chromatography (AEX) (charge heterogeneity, main peak, total acidic variants, total basic variants), non-reduced capillary electrophoresis sodium dodecyl sulfate (CE-SDS), poloxamer content, pH, micro-flow imaging (MFI), high accuracy liquid particle counting (HIAC), and functionality testing.

Results show chemical and physical stability is sufficient to allow for storage of at least 24 months at 5° C. and at least 2 weeks of storage at temperatures up to 30° C. without loss of stability.

```
Sequences
SEQ ID NO: 1
                  10 20 30 40 50 60
FVNQHLCGSHLVEALELVCGERGFHYGGGGGGSGGGGGIVEQCCTSTCSLDQLENYCGGG
                  70 80 90 100 110 120
GGQGGGGQGGGGQGGGGGECPPCPAPPVAGPSVFLFPPKPKDTLMISRTPEVTCVVVDVS
                  130 140 150 160 170 180
HEDPEVQFNWYVDGVEVHNAKTKPREEQFNSTFRVVSVLIVVHQDWLNGKEYKCKVSNKG
                  190 200 210 220 230 240
LPAPIEKTISKTKGQPREPQVYTLPPSREEMTKNQVSLTCLVKGFYPSDIAVEWESNGQP
                  250 260 270 280 290
ENNYKTTPPMLDSDGSFFLYSKLTVDKSRWQQGNVFSCSVMHEALHNHYTQKSLSLSPG
```

1. A method of providing glycemic control in a patient in need thereof with type 2 diabetes (T2D) comprising: administering to said patient once-weekly a fixed dose of insulin efsitora alfa selected from the group consisting of 100, 150, 250 and 400 U.

2. The method of claim 1, wherein the first dose of insulin efsitora alfa administered to the patient is 100 U.

3. The method of claim 2 wherein the patient is administered the same dose for at least 4 weeks, and wherein the dose is increased when the patient needs additional glycemic control.

4. The method of claim 1 wherein the patient's dose is increased if the patient's fasting glucose (FG) is >130 mg/dL after treatment with a first fixed dose for at least 4 weeks.

5. The method of claim 4, wherein the patient's dose is only increased if the patient had 0 episodes of blood glucose <70 mg/dL.

6. The method of claim 5 wherein the patient's dose is decreased if the patient's FG is <80 mg/dL.

7. A method of improving glycemic control in a patient in need thereof with type 2 diabetes (T2D) comprising:
a) administering to said patient a 100 U of insulin efsitora alfa once-weekly;
b) increasing the dose to 150 U of insulin efsitora alfa once-weekly after at least 4 weeks on the 100 U dose;
c) increasing the dose to 250 U of insulin efsitora alfa once-weekly after at least 4 weeks on the 150 U dose; and
d) increasing the dose to 400 U of insulin efsitora alfa once-weekly after at least 4 weeks on the 250 U dose.

8. The method of claim 7 wherein steps b) through d) are performed to reduce the patient's FG.

9. The method of claim 7 wherein steps b) through d) are performed when the patient's FG is >130 mg/dL.

10. The method of claim 9 wherein steps b) through d) are only performed when the patient had 0 episodes of blood glucose <70 mg/dL.

11. The method of claim 10 wherein the patient's dose is decreased to the previous lower dose if the patient's FG is <80 mg/dL.

12. The method of claim 11, wherein the patient discontinues treatment if the patient experiences one or more episodes of nocturnal hypoglycemia or 2 or more episodes of hypoglycemia.

13. The method of claim 1 wherein the patient is insulin-naïve.

14. The method of claim 13 wherein patient has uncontrolled hyperglycemia.

15. The method of claim 13, wherein the patient has HbA1c between 7 and 10%.

16. The method of claim 15, wherein the patient is receiving 2 or more oral antihyperglycemic medications.

17. The method of claim 15 wherein the patient is receiving a GLP-1 receptor agonist.

18. The method of claim 1 wherein insulin efsitora alfa is in administered in an aqueous composition comprising: phosphate in a concentration between about 5 and 10 mM; and glycerol in a concentration between about 15 and 35 mM; and having a pH between about 5.5 and 7.5.

19. The method of claim 18, wherein the composition further comprises poloxamer 188 in a concentration between about 0.1 to about 0.5 mg/mL.

20. The method of claim 19 wherein the phosphate is in a concentration of about 10 mM; glycerol is in a concentration of about 25 mM; and poloxamer 188 is in a concentration of about 0.4 mg/mL; and wherein the pH of the composition is about 6.5.

21. An aqueous pharmaceutical composition comprising:
a) a fixed dose of insulin efsitora alfa in an amount selected from the group consisting of 100, 150, 250 and 400 U;
b) phosphate in a concentration between about 5 and 10 mM; and
c) glycerol in a concentration between about 15 and 35 mM;
and having a pH between about 5.5 and 7.5.

22. The composition of claim 21, further comprising poloxamer 188 in a concentration between about 0.1 to about 0.5 mg/mL.

23. The composition of claim 22 wherein the phosphate is in a concentration of about 10 mM; glycerol is in a concentration of about 25 mM; and poloxamer 188 is in a concentration of about 0.4 mg/mL; and wherein the pH of the composition is about 6.5.

24. A method of providing glycemic control in a patient in need thereof with type 2 T2D comprising administering to said subject the composition of claim 23.

25. The method of claim 1 wherein the fixed dose of insulin efsitora alfa is provided in a single-use autoinjector.

26. A single-use autoinjector for use in the method of claim 1.

27. A single-use autoinjector comprising the composition of claim 23.

28. The method of claim 1 wherein the method comprises improving glycemic control in the patient.

29-51. (canceled)
